(19) United States
(12) Patent Application Publication
(10) Pub. No.: US 2006/0247780 A1
(43) Pub. Date: Nov. 2, 2006

(54) Expandable artificial disc and associated methods and instrumentation

(76) Inventor: Jeffrey K. Bert, Coosbay, OR (US)

Correspondence Address: KRIEG DEVAULT LLP, ONE INDIANA SQUARE, SUITE 2800, INDIANAPOLIS, IN, 46204-2709, US

(21) Appl. No.: 11/115,762

(22) Filed: Apr. 27, 2005

(51) Int. Cl. A61F 2/44; A61B 17/16

(52) U.S. Cl. 623/17.16; 623/17.12; 606/79

(57) ABSTRACT

An artificial disc including an inner expandable region and an outer peripheral region. The expandable region is transitionable to an expanded configuration having an expanded height that is greater than the intervertebral disc space height, and with the peripheral region having a height substantially equal to the intervertebral disc space height. In one embodiment, the peripheral region has an outer profile substantially corresponding to the size and shape of the intervertebral disc space. In another embodiment, the expandable region comprises a non-porous flexible bladder that is transitionable to the expanded configuration via introduction of an expansion media into an interior chamber of the bladder, with the artificial disc further including an inlet port for introducing expansion media into the interior chamber, and an outlet port for removing expansion media from the interior chamber. In a further embodiment, a vacuum source is positioned in communication with the interior chamber to facilitate removal of expansion media therefrom.

### FIELD OF THE INVENTION

The present invention relates generally to the field of intervertebral implants, and more particularly relates to an expandable artificial disc and associated methods and instrumentation.

### BACKGROUND

With regard to spinal deformities, the intervertebral disc space height between adjacent vertebrae can be lacking or abnormal due to the condition of the disc space and/or due to conditions resulting from a surgical procedure. Intervertebral implants have been developed to restore the natural height of the disc space and which provide the ability to adjust the height of the implant subsequent to insertion within the disc space. However, such adjustments can require mechanical manipulation of cumbersome and intricate instruments within the disc space to correspondingly adjust the height of the implant. Such adjustments can also result in a non-uniform distribution of loads on the vertebral endplates at their interface with respective surfaces of the implant.

Thus, there remains a need for an improved intervertebral implant that is expandable within the disc space. The present invention satisfies this need and provides other benefits and advantages in a novel and unobvious manner.

### SUMMARY

The present invention relates generally to an expandable artificial disc and associated methods and instrumentation. While the actual nature of the invention covered herein can only be determined with reference to the claims appended hereto, certain forms of the invention that are characteristic of the preferred embodiments disclosed herein are described briefly as follows.

In one form of the present invention, an artificial disc is provided for implantation within an intervertebral disc space between adjacent vertebrae, including an inner expandable region and an outer peripheral region. The inner expandable region is transitionable between an initial configuration and an expanded configuration, with the expanded configuration having an expanded height that is greater than a height of the intervertebral disc space. The outer peripheral region has a height that is substantially equal to the height of the intervertebral disc space and has an outer profile substantially corresponding to the size and shape of the intervertebral disc space.

In another form of the present invention, an artificial disc is provided for implantation within an intervertebral disc space between adjacent vertebrae, including an inner expandable region and an outer peripheral region. The inner expandable region comprises a non-porous flexible bladder and is transitionable between an initial configuration and an expanded configuration via the introduction of an expansion media into the non-porous flexible bladder, with the expanded configuration having an expanded height that is greater than a height of the intervertebral disc space. The outer peripheral region has a height that is substantially equal to the height of the intervertebral disc space.

In another form of the present invention, an artificial disc is provided for implantation within an intervertebral disc space between adjacent vertebrae, including an inner expandable region and an outer peripheral region. The inner expandable region defines an interior chamber and is transitionable from an initial configuration to an expanded configuration via introduction of an expansion media into the interior chamber, with the expanded configuration having an expanded height that is greater than a height of the intervertebral disc space. The outer peripheral region has a height that is substantially equal to the height of the intervertebral disc space. The artificial disc includes an inlet port in communication with the interior chamber and which is adapted to introduce expansion media into the interior chamber, and an outlet port in communication with the interior chamber and which is adapted for removal of at least a portion of the expansion media from the interior chamber.

In another form of the present invention, a method is provided for implanting an artificial disc within an intervertebral disc space between adjacent vertebrae. The method includes the step of providing an artificial disc including an inner expandable region and an outer peripheral region, with the expandable region comprising a non-porous flexible bladder that is transitionable from an initial configuration to an expanded configuration, and with the peripheral region having upper and lower surfaces defining a height substantially equal to the height of the intervertebral disc space. The method further includes the steps of forming a recess in a vertebral endplate of at least one of the adjacent vertebrae, inserting the artificial disc into the intervertebral disc space with the upper and lower surfaces of the peripheral region facing respective ones of the adjacent vertebrae and with the inner region generally aligned with the recess in the vertebral endplate, and introducing an expansion media into the non-porous flexible bladder to transition the inner region to the expanded configuration with the expanded inner region positioned within the recess in the vertebral endplate.

In another form of the present invention, an instrument is provided for preparing first and second vertebrae for receipt of an artificial disc within the intervertebral disc space between the vertebrae. The instrument includes a distal end portion positionable within the intervertebral disc space, and a handle portion extending from the distal end portion and positionable outside of the intervertebral disc space. The distal end portion includes a ball-shaped cutting portion and an annular ring portion extending about a periphery of the ball-shaped cutting portion. The ball-shaped cutting portion includes a first cutting element configured to form a first spherical-shaped recess in the first vertebra, and a second cutting element configured to form a second spherical-shaped recess in the second vertebra.

It is one object of the present invention to provide an improved artificial disc and associated methods and instrumentation. Further objects, features, advantages, benefits, and aspects of the present invention will become apparent from the drawings and description contained herein.

### BRIEF DESCRIPTION OF THE DRAWINGS

FIG. 1 is a perspective view of an expandable artificial disc according to one form of the present invention.

FIG. 2 is a cross sectional view of the artificial disc illustrated in FIG. 1, as shown in an initial, non-expanded configuration.

FIG. 3 is a cross sectional view of the artificial disc taken along line 3-3 of FIG. 1, as shown in an expanded configuration.

FIG. 4 is a lateral view of a spinal column with a first spherical-shaped recess formed in the inferior endplate of an upper vertebra, and a second spherical-shaped recess formed in the superior endplate of a lower vertebra.

FIG. 5 is a top plan view of a reamer instrument according to one form of the present invention for forming spherical-shaped recesses in the upper and lower vertebrae.

FIG. 6 is a side view of the reamer instrument illustrated in FIG. 5.

FIG. 7 is a lateral view of the spinal column illustrating insertion of the artificial disc into the intervertebral disc space between the upper and lower vertebrae while in the initial, non-expanded configuration illustrated in FIG. 2.

FIG. 8 is a lateral view of the spinal column illustrating expansion of the expandable artificial disc within the intervertebral disc space between the upper and lower vertebrae to the expanded configuration illustrated in FIG. 3.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, and that alterations and further modifications to the illustrated devices and/or further applications of the principles of the invention as illustrated herein are contemplated as would normally occur to one skilled in the art to which the invention relates.

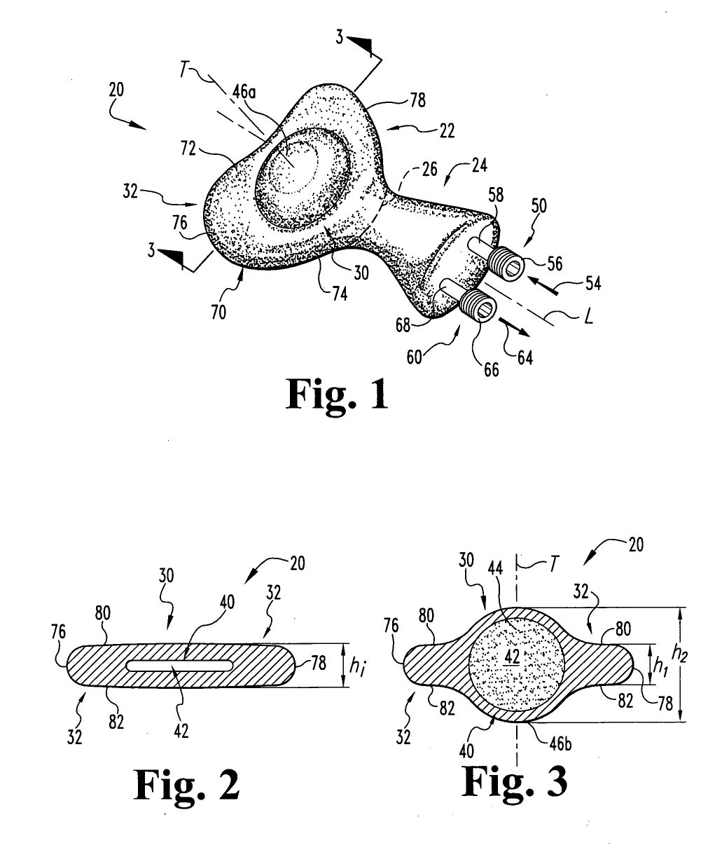

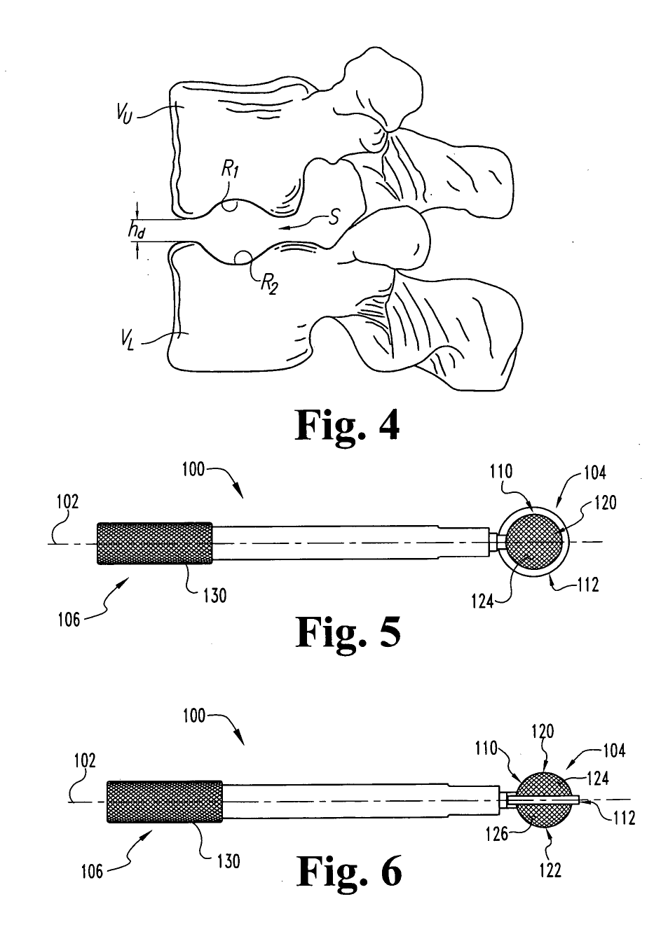

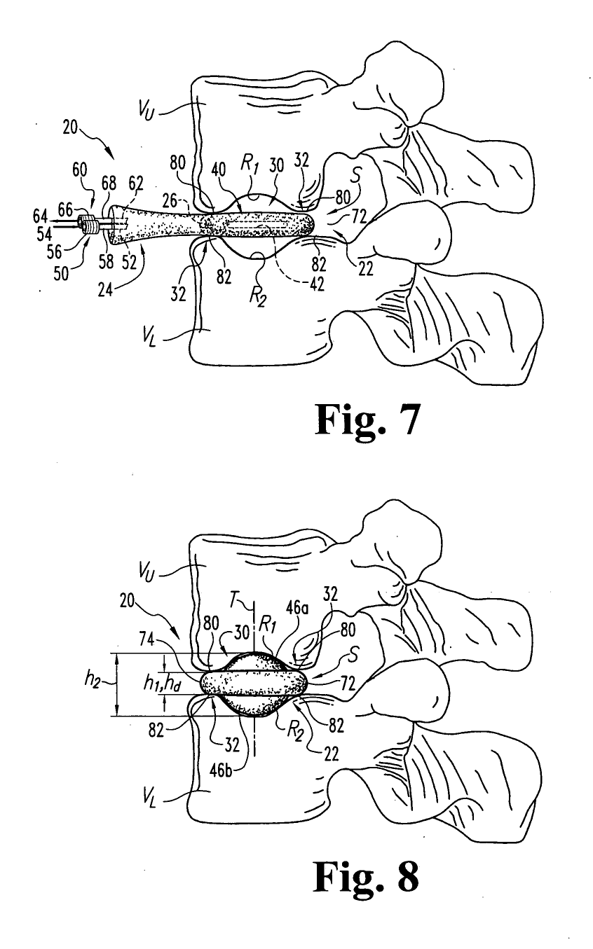

Referring to FIGS. 1-3, shown therein is an expandable artificial disc 20 according to one form of the present invention. The expandable artificial disc 20 extends generally along a longitudinal axis L and is transitionable between an initial configuration (FIG. 2) and an expanded configuration (FIGS. 1 and 3), with the expansion occurring generally along a transverse axis T. In the illustrated embodiment, the artificial disc 20 generally comprises a main body portion 22 and an extension portion 24. The main body portion 22 is sized and shaped for insertion within an intervertebral disc space S between upper and lower vertebrae V_{U}, V_{L}, with the extension portion 24 extending outside of the intervertebral disc space S (FIG. 7). The extension portion 24 is selectively removable from the main body portion 22 generally along a separation line 26 subsequent to transitioning of the artificial disc 20 to the expanded configuration (FIG. 8). However, it should be understood that other configurations of the artificial disc 20 are also contemplated as falling within the scope of the present invention, including configurations wherein the artificial disc 20 includes the main body portion 22 without the extension portion 24. Further details regarding the structure and function of the extension portion 24 will be discussed below.

In the illustrated embodiment of the invention, the main body portion 22 comprises an inner expandable region 30 and an outer peripheral region 32 extending about the inner region 30. The outer peripheral region 32 has a height h, that is substantially equal to the intervertebral disc space height h_{d} between the upper and lower vertebrae V_{U}, V_{L}(FIG. 8). As shown in FIGS. 2 and 3, the inner region 30 is transitionable between an initial configuration and an expanded configuration, respectively, with the expanded configuration having an expanded height h₂ that is greater than the height h₁ of the peripheral region 32 and the intervertebral disc space height h_{d}, the details of which will be discussed below. Additionally, in one embodiment, the outer peripheral region 32 does not appreciably expand or change shape as the inner region 30 is transitioned to the expanded configuration. As a result, the height h₁ of the peripheral region 32 remains substantially unchanged as the inner region 30 is transitioned to the expanded configuration. Additionally, the inner region 30 preferably has an initial, non-expanded height hᵢ (FIG. 2) that is substantially equal to or possibly less than the height h₁ of the peripheral region 32. As should be appreciated, providing the inner region 30 with an initial, non-expanded height hᵢ that is substantially equal to or possibly less than the height h₁ of the peripheral region 32 provides the artificial disc 20 with a lower vertical profile to facilitate insertion of the artificial disc 20 into the intervertebral disc space S and to minimize distraction of the upper and lower vertebrae V_{U}, V_{L} to accommodate such insertion.

In one aspect of the invention, the inner expandable region 30 of the artificial disc 20 comprises a non-porous flexible bladder 40 defining an interior chamber 42, with the inner region 30 being transitioned to the expanded configuration via introduction of an expansion media 44 (FIG. 3) into the interior chamber 42 of the flexible bladder 40. When transitioned to the expanded configuration, the inner region 30 defines a pair of upper and lower spherical-shaped protrusions 46a, 46b extending in opposite directions and which are positionable within a pair of spherical-shaped recesses R₁, R₂ formed in respective ones of the upper and lower vertebrae V_{U}, V_{L}(FIG. 8). However, it should be understood that other shapes and configurations of the expanded inner region 30 are also contemplated including, for example, cylindrical, elliptical or oval configurations, or any other suitable shape or configuration. Additionally, it should be understood that the expanded inner region 30 need not necessarily define a pair of spherical-shaped protrusions, but may alternatively define a single protrusion positionable within a corresponding recess formed in one of the upper and lower vertebrae V_{U}, V_{L}. In the illustrated embodiment, the outer surfaces of the spherical-shaped protrusions 46a, 46b are substantially smooth. However, in other embodiments, the outer surfaces of the spherical-shaped protrusions 46a, 46b may define a number of surface projections, such as spikes or teeth, or surface roughening to aid in gripping the upper and lower vertebrae V_{U}, V_{L}.

In one embodiment of the invention, the inner expandable region 30 is formed of a flexible polymeric material. However, other materials that exhibit sufficient flexibility to facilitate transitioning of the inner region 30 from the initial configuration illustrated in FIG. 2 to the expanded configuration illustrated in FIG. 3 are also contemplated including, for example, synthetic materials, fibrous materials, reinforced composite materials, shape-memory alloy materials, stainless steel and stainless steel alloys, titanium and titanium alloys, cobalt chrome alloys, ceramic materials, bone or a bone substitute materials, or any other suitable bio-compatible material. In another embodiment of the invention, the outer peripheral region 32 is also formed of a polymeric material. However, other materials that exhibit sufficient strength to resist compression loads exerted by the upper and lower vertebrae V_{U}, V_{L} are also contemplated including, for example, synthetic materials, fibrous materials, reinforced composite materials, shape-memory alloy materials, stainless steel and stainless steel alloys, titanium and titanium alloys, cobalt chrome alloys, ceramic materials, bone or a bone substitute materials, or any other suitable bio-compatible material. In a specific embodiment, the peripheral region 32 is formed of the same material as the inner region 30. However, it is also contemplated that the inner region 30 and the peripheral region 32 may be formed from different materials. In another specific embodiment, the peripheral region 32 is formed integral with the inner region 30 so as to define a single-piece, unitary artificial disc 20. However, it is also contemplated that the inner region 30 and the peripheral region 32 may be formed separately and subsequently assembled or attached to one another to form the artificial disc 20.

In another embodiment of the invention, the expansion media 44 is at least partially comprised of a flowable material such as, for example, a fluidic material. In a further embodiment, the expansion media 44 is injectable under pressure into the interior chamber 42 of the flexible bladder 40. In a specific embodiment, the expansion media 44 used to expand the inner region 30 comprises a mixture of a resin material and a catalyst material, with the materials interacting with one another to form a hardened material. In another specific embodiment, the expansion media 44 comprises a polymer-based material. In yet another specific embodiment, the expansion media 44 comprises a thixotropic gel. In a further specific embodiment, the expansion media 44 comprises a bone cement. In still another specific embodiment, the expansion media 44 comprises a saline solution or water. In a further embodiment of the invention, the expansion media 44 may comprise a radio opaque material. However, it should be understood that other types of expansion media are also contemplated as being suitable for use in association with the present invention including, for example, a gaseous media such as compressed air, or a solid media such as beads or bone graft.

In another aspect of the invention, the artificial disc 20 includes an inlet port 50 for introducing the expansion media 44 into the interior chamber 42 of the flexible bladder 40, and an outlet port 60 for removing at least a portion of the expansion media 44 from the interior chamber 42. The inlet port 50 is positioned in communication with the interior chamber 42 via an inlet passage 52 (FIG. 7). The inlet port 50 is in turn positioned in communication with a supply source 54 for providing expansion media 44 to the interior chamber 42 of the flexible bladder 40. The outlet port 60 is positioned in communication with the interior chamber 42 via an outlet passage 62. In one embodiment, the outlet port 60 is positioned in communication with a vacuum source 64 to facilitate removal of at least a portion of the expansion media 44 from the interior chamber 42 of the flexible bladder 40. However, it should be understood that the outlet port 60 does not necessarily have to be positioned in communication with a vacuum source to remove material from the flexible bladder 40, but may instead be exposed to atmospheric pressure or even a slightly positive pressure, with the fluid pressure associated with the supply source 54 serving to force a portion of the expansion media 44 out of the interior chamber 42. It should also be understood that other embodiments of the invention are also contemplated that do not include an outlet port 60.

In the illustrated embodiment of the invention, the inlet port 50 includes a threaded stem portion 56 configured for threading engagement with a corresponding threaded portion of a supply port (not shown) associated with the supply source 54, and a tubular portion 58 positioned in communication with the inlet passage 52. Similarly, the outlet port 60 includes a threaded stem portion 66 configured for threading engagement with a corresponding threaded portion of a removal port (not shown) associated with the vacuum source 64, and a tubular portion 68 positioned in communication with the outlet passage 62. However, it should be understood that other types and configurations of the inlet and outlet ports 50, 60 are also contemplated for use in association with the present invention. As shown in FIG. 1, in the illustrated embodiment of the invention, the inlet and outlet ports 50, 60 are positioned adjacent the distal end of the extension portion 24 of the artificial disc 20. However, in other embodiments, the inlet and outlet ports 50, 60 may be positioned adjacent other portions of the artificial disc 20, including the main body portion 22.

As discussed above and as illustrated in FIG. 7, the extension portion 24 is disposed outside of the intervertebral disc space S when the artificial disc 20 is positioned between the upper and lower vertebrae V_{U}, V_{L}. Accordingly, the inlet and outlet ports 50, 60 are also positioned outside of the intervertebral disc space S to provide convenient and unimpeded access to the ports 50, 60 to facilitate connection to and/or disconnection from the supply source 54 and the vacuum source 64. As indicated above, once the artificial disc 20 is fully transitioned to the expanded configuration, the extension portion 24 may removed from the main body portion 22, generally along the separation line 26, with the main body portion 22 positioned entirely within the intervertebral disc space S. The inlet passage 52 and the outlet passage 62 may be closed off via plug members to retain the expansion media 44 within the interior chamber 42, or may be configured to self-seal upon removal of the extension portion 24 such as, for example, via the use of a self-sealing membrane. Alternatively, a valve member may be pre-positioned within each of the inlet and outlet passages 52, 62 to permit passage of the expansion media 44 therethrough while retaining the expansion media 44 within the interior chamber 42 following removal of the extension portion 24.

In a further aspect of the invention, the peripheral region 32 has an outer profile 70 (FIG. 1) that substantially corresponds to the size and shape of the intervertebral disc space S. In one embodiment, the outer profile 70 defines a non-circular shape. In another embodiment, the outer profile 70 is non-symmetrical relative to a coronal plane extending between the adjacent vertebrae (e.g., along line 3-3). In one specific embodiment, the outer profile 70 is substantially kidney-shaped. In another specific embodiment, the outer profile 70 is substantially D-shaped. In a further specific embodiment, the peripheral region 32 of the artificial disc includes a posterior surface 72 positionable adjacent a posterior region of the intervertebral disc space, with the posterior surface 72 defining a concave curvature. The peripheral region 32 also includes an anterior surface 74 and a pair of lateral surfaces 76, 78 extending between the posterior and anterior surfaces 72, 74, with each of the lateral surfaces 76, 78 and the anterior surface 74 defining a convex curvature. However, it should be understood that other shapes and configurations of the outer profile 70 of the peripheral region 32 are also contemplated as falling within the scope of the present invention.

Additionally, the peripheral region 32 includes a superior surface 80 and an inferior surface 82 which bear against the endplates of the upper and lower vertebrae V_{U}, V_{L}(FIG. 8) when the artificial disc 20 is positioned within the intervertebral disc space S to provide support and resistance to a substantial amount of the compressive forces exerted onto the artificial disc 20. As should be appreciated, a vertebra is comprised of a hard cortical bone material extending about the outer region of the vertebral body, and a softer cancellous or spongiose bone material positioned interior to the cortical bone material. As should further be appreciated, since the outer profile 70 of the peripheral region 32 substantially corresponds to the size and shape of the intervertebral disc space S, the superior and inferior surfaces 80, 82 of the peripheral region 32 bear against the outer cortical rim/apophyseal ring region of the upper and lower vertebrae V_{U}, V_{L}, thereby tending to increase stability of the artificial disc 20 and minimizing subsidence into the relatively soft cancellous bone. In the illustrated embodiment, the superior and inferior surfaces 80, 82 are substantially smooth. However, in other embodiments, the superior and inferior surfaces 80, 82 may define a number of surface projections, such as spikes or teeth, or surface roughening to aid in gripping the upper and lower vertebrae V_{U}, V_{L}. Additionally, although the superior and inferior surfaces 80, 82 are illustrated as having a substantially flat or planar configuration, it should be understood that the surfaces 80, 82 may be curved or contoured. Furthermore, although the superior and inferior surfaces 80, 82 are illustrated as being arranged substantially parallel to one another, it should be understood that the surfaces 80, 82 may be tapered or angled relative to one another to accommodate for lordosis between the upper and lower vertebrae V_{U}, V_{L}.

Having described various elements and features associated with the artificial disc 20, reference will now be made to a method for implanting the artificial disc 20 within the intervertebral disc space S according to one form of the present invention. However, it should be understood that other implantation techniques and procedures are also contemplated, and that the following method in no way limits the scope of patent protection sought for the present invention.

Initially, the portion of the spinal column to be treated is identified and accessed from a posterior approach using known surgical techniques. However, it should be understood that other surgical approaches are also contemplated including, for example, an anterior approach or a lateral approach. It should further be understood that the artificial disc 20 may be used to treat any region of the spinal column, including the cervical, thoracic, lumbar or sacral regions of the spine. At least a portion of the natural intervertebral disc is removed via a total or partial discectomy to provide an intervertebral disc space S for receipt of the artificial disc 20 between the upper and lower vertebrae V_{U}, V_{L}. The intervertebral disc space S may be distracted to a height h_{d} that is substantially equal to the natural disc space height.

Prior to insertion of the artificial disc 20 into the disc space S, the endplates of the upper and lower vertebrae V_{U}, V_{L} are prepared using various cutting tools and instrumentation including, for example, the cutting instrument 100 illustrated in FIGS. 5 and 6. In the illustrated embodiment, the cutting instrument 100 extends generally along a longitudinal axis 102 and includes a distal end portion 104 positionable within the intervertebral disc space S and a handle portion 106 extending from the distal end portion 102 and positioned outside of the intervertebral disc space S.

In one embodiment, the distal end portion 104 includes a ball-shaped cutting portion 110 and an annular ring portion 112 extending about a periphery of the ball-shaped cutting portion 110. The ball-shaped cutting portion 110 includes a first cutting element portion 120 positioned on one side of the ring portion 112 to form a first spherical-shaped recess R₁ in the upper vertebra V_{U}, and a second cutting element portion 122 positioned on the opposite side of the ring portion 112 to form a second spherical-shaped recess R₂ in the lower vertebra V_{L}. The first and second cutting element portions 120, 122 include upper and lower cutting surfaces 124, 126 which, when rotated relative to the longitudinal axis 102, cut into the upper and lower vertebrae V_{U}, V_{L} to form the spherical-shaped recess R₁, R₂. In one embodiment, rotation of the upper and lower cutting surfaces 124, 126 is accomplished via rotation of the handle portion 106 about the longitudinal axis 102. In a further embodiment, the outer surface 130 of the handle portion 106 is roughened such as, for example, by knurling to aid in manual rotation of the handle portion 106 to correspondingly rotate the upper and lower cutting surfaces 124, 126.

In the illustrated embodiment, the recesses R₁, R₂ formed in the upper and lower vertebrae V_{U}, V_{L} each have a spherical configuration sized for receipt of the upper and lower spherical-shaped protrusions 46a, 46b defined by the expanded configuration of the artificial disc 20. However, it should be understood that other shapes and configurations of the recesses R₁, R₂ are also contemplated including, for example, cylindrical, elliptical or oval configurations, or any other suitable shape or configuration. In one embodiment, the recesses R₁, R₂ are formed in the cancellous region of the upper and lower vertebrae V_{U}, V_{L}, with the cortical rim/apophyseal ring region of the vertebral endplates remaining substantially intact.

Following preparation of the vertebral endplates, the artificial disc 20 is inserted into the intervertebral disc space S with the superior and inferior surfaces 80, 82 of the peripheral region 32 facing respective ones of the upper and lower vertebrae V_{U}, V_{L}, and with the inner region 30 generally aligned with the spherical-shaped recesses R₁, R₂. The superior and inferior surfaces 80, 82 of the peripheral region 32 are positioned to bear against the cortical rim/apophyseal ring region of the upper and lower vertebrae V_{U}, V_{L}, respectively, to resist compression loads, to increase the overall stability of the artificial disc 20, and to minimize subsidence into the relatively softer cancellous bone. In one embodiment, the artificial disc 20 may be inserted into the intervertebral disc space S in a minimally invasive manner (i.e., through a small access portal) via the use of endoscopic equipment, a small diameter tube or cannula, or by other minimally invasive surgical techniques. However, it should be understood that the artificial disc 20 may also be inserted into the intervertebral disc space S using conventional surgical methods and techniques. Notably, since the artificial disc 20 is inserted into the disc space while in a non-expanded configuration having an initial maximum height h₁ that is substantially equal to or somewhat less than the disc space height h_{d}, over distraction of the intervertebral disc space S is avoided and neural distraction is minimized.

Following insertion of the artificial disc 20 into the intervertebral disc space S, the inner region 30 is expanded generally along the transverse axis T via introduction of the expansion media 44 into the interior chamber 42 of the flexible bladder 40. As illustrated in FIG. 8, transitioning of the inner region 30 to the expanded configuration results in outward deformation of the flexible bladder 40 to provide a pair of spherical-shaped protrusions 46a, 46b extending in opposite directions relative to the outer peripheral region 32. Once the artificial disc 20 is fully transitioned to the expanded configuration, the extension portion 24 may be selectively removed from the main body portion 22 generally along the separation line 26. As illustrated in FIG. 8, upon the removal of the extension portion 24, the remaining body portion 22 of the artificial disc 20 is preferably entirely disposed within the intervertebral disc space S. In one embodiment, the extension portion 24 is cut off from the main body portion 22 generally along the separation line 26. However, other techniques for selectively removing the extension portion 24 from the main body portion 22 are also contemplated including, for example, a break-off or tear-off technique, or any other removal technique that would occur to one of skill in the art.

When transitioned to the expanded configuration, the inner region 30 has an expanded height h₂ that is greater than the intervertebral disc space height h_{d}. Additionally, transitioning of the artificial disc 20 to the expanded configuration illustrated in FIG. 8 positions the spherical-shaped protrusions 46a, 46b within the spherical-shaped recesses R₁, R₂, which in turn tends to increase the overall stability of the artificial disc 20 and also tends to reduce the likelihood of migration and possible expulsion of the artificial disc 20 from the intervertebral disc space S. Although it is contemplated that the artificial disc 20 may be used without any other means of fixation, it should be understood that supplemental external intravertebral fixation elements and/or stabilization techniques may be used if excessive residual instability is encountered following insertion and expansion of the artificial disc 20 within the intervertebral disc space S. Moreover, as the inner region 30 expands, the spherical-shaped protrusions 46a, 46b are compressed against the opposing surfaces of the upper and lower vertebrae V_{U}, V_{L}, which in turn may cause the vertebrae V_{U}, V_{L} to slightly spread apart. In instances where the annulus of the intervertebral disc remains intact, expansion of the inner region 30 tightens and possibly stretches the disc annulus, thereby providing additional stabilization of the upper and lower vertebrae V_{U}, V_{L}.

As should be appreciated, if removal of the artificial disc 20 from the intervertebral disc space S is required due to non-optimal placement or for other reasons, the artificial disc 20 can be transitioned from the expanded configuration back toward the initial configuration (FIG. 2) by simply removing some or all of the expansion media 44 from the interior chamber 42 of the flexible bladder 40 via activation of the vacuum source 64. In the event that the artificial disc 20 is over-expanded or requires partial contraction, the vacuum source 64 may be activated to remove a portion of the expansion media 44 from the interior chamber 42 of the flexible bladder 40. As should be appreciated, removal of a portion of the expansion media 44 from the interior chamber 42 of the flexible bladder 40 in turn causes the spherical-shaped protrusions 46a, 46b to contract and withdraw from the recesses R₁, R₂ in the upper and lower vertebrae V_{U}, V_{L}, thereby allowing for revision or possible removal of the artificial disc 20 from the intervertebral disc space S.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. An artificial disc for implantation within an intervertebral disc space between adjacent vertebrae, comprising:
an inner expandable region transitionable between an initial configuration and an expanded configuration, said expanded configuration having an expanded height greater than a height of the intervertebral disc space; and
an outer peripheral region extending about said inner expandable region, said peripheral region having a height substantially equal to the height of the intervertebral disc space and having an outer profile substantially corresponding to the size and shape of the intervertebral disc space.

2. The artificial disc of claim 1, wherein said outer profile of said peripheral region has a non-circular shape.

3. The artificial disc of claim 2, wherein said outer profile of said peripheral region is non-symmetrical relative to a coronal plane extending between the adjacent vertebrae.

4. The artificial disc of claim 1, wherein said outer profile of said peripheral region is substantially kidney-shaped.

5. The artificial disc of claim 1, wherein said outer peripheral region includes a posterior surface positionable adjacent a posterior region of the intervertebral disc space, said posterior surface defining a concave curvature.

6. The artificial disc of claim 5, wherein said peripheral region includes an anterior surface and a pair of lateral surfaces extending between said posterior and anterior surfaces, each of said lateral surfaces and said anterior surface defining a convex curvature.

7. The artificial disc of claim 1, wherein said outer profile of said peripheral region is substantially D-shaped.

8. The artificial disc of claim 7, wherein said peripheral region includes a posterior surface positionable adjacent a posterior region of the intervertebral disc space, said posterior surface defining a concave curvature.

9. The artificial disc of claim 1, wherein said expanded configuration of said expandable region defines a pair of spherical-shaped protrusions extending in opposite directions and positionable within spherical-shaped recesses formed in respective ones of the adjacent vertebrae.

10. The artificial disc of claim 1, wherein said peripheral region does not appreciably expand as said expandable region is transitioned to said expanded configuration.

11. The artificial disc of claim 1, wherein said an expandable region comprises a non-porous flexible bladder, said expandable region being transitioned to said expanded configuration by introduction of an expansion media into said non-porous flexible bladder.

12. The artificial disc of claim 11, wherein said expansion media is selected from the group consisting of a resin/catalyst mixture, a polymer-based material, a bone cement, a saline solution and water.

13. The artificial disc of claim 11, further comprising:
an inlet port in communication with said non-porous flexible bladder for introduction of said expansion media into said non-porous flexible bladder; and
an outlet port in communication with said non-porous flexible bladder for removal of at least a portion of said expansion media from said non-porous flexible bladder.

14. The artificial disc of claim 13, further comprising a vacuum source in communication with said outlet port to facilitate said removal of said at least a portion of said expansion media from said non-porous flexible bladder.

15. An artificial disc for implantation within an intervertebral disc space between adjacent vertebrae, comprising:
an inner expandable region comprising a non-porous flexible bladder that is transitionable between an initial configuration and an expanded configuration by introduction of an expansion media into said non-porous flexible bladder, said expanded configuration having an expanded height greater than a height of the intervertebral disc space; and
an outer peripheral region extending about said inner expandable region, said peripheral region having a height substantially equal to the height of the intervertebral disc space.

16. The artificial disc of claim 15, wherein said expansion media at least partially comprises a fluid.

17. The artificial disc of claim 15, wherein said expansion media comprises a resin/catalyst mixture.

18. The artificial disc of claim 15, wherein said expansion media comprises a polymer-based material.

19. The artificial disc of claim 15, wherein said expansion media comprises bone cement.

20. The artificial disc of claim 15, wherein said expansion media comprises a saline solution.

21. The artificial disc of claim 15, further comprising:
an inlet port in communication with said non-porous flexible bladder for introduction of said expansion media into said non-porous flexible bladder; and
an outlet port in communication with said non-porous flexible bladder for removal of at least a portion of said expansion media from said non-porous flexible bladder.

22. The artificial disc of claim 21, further comprising a vacuum source in communication with said outlet port to facilitate said removal of said at least a portion of said expansion media from said non-porous flexible bladder.

23. The artificial disc of claim 15, wherein said peripheral region has an outer profile substantially corresponding to the size and shape of the intervertebral disc space.

24. The artificial disc of claim 23, wherein said outer profile of said peripheral region is substantially kidney-shaped.

25. The artificial disc of claim 23, wherein said peripheral region includes a posterior surface positionable adjacent a posterior region of the intervertebral disc space, said posterior surface defining a concave curvature.

26. The artificial disc of claim 15, wherein said expanded configuration of said expandable region defines a pair of spherical-shaped protrusions extending in opposite directions and positionable within spherical-shaped recesses formed in respective ones of the adjacent vertebrae.

27. The artificial disc of claim 15, wherein said peripheral region does not appreciably expand as said inner region is transitioned to said expanded configuration.

28. The artificial disc of claim 15, wherein said height of said peripheral region remains substantially unchanged as said expandable region is transitioned to said expanded configuration.

29. The artificial disc of claim 28, wherein said expandable region has an initial height when in said initial configuration that is substantially equal to said substantially unchanged height of said peripheral region.

30. An artificial disc for implantation within an intervertebral disc space between adjacent vertebrae, comprising:
an inner expandable region defining an interior chamber and being transitionable from an initial configuration to an expanded configuration by introduction of an expansion media into said interior chamber, said expanded configuration having an expanded height greater than a height of the intervertebral disc space;
an outer peripheral region extending about said inner expandable region and having a height substantially equal to the height of the intervertebral disc space;
an inlet port in communication with said interior chamber and adapted for said introduction of said expansion media into said interior chamber; and
an outlet port in communication with said interior chamber and adapted for removal of at least a portion of said expansion media from said interior chamber.

31. The artificial disc of claim 30, further comprising a vacuum source in communication with said outlet port to facilitate said removal of said at least a portion of said expansion media from said interior chamber.

32. The artificial disc of claim 30, wherein said inlet port includes a first threaded portion configured for threading engagement with a corresponding threaded portion of a supply port, said supply port in communication with a supply source of said expansion media, said outlet port including a second threaded portion configured for threading engagement with a corresponding threaded portion of a removal port, said removal port in communication with a container for receiving said at least a portion of said expansion media removed from said interior chamber.

33. The artificial disc of claim 30, further comprising an extension extending outside of the intervertebral disc space when the artificial disc is positioned between the adjacent vertebrae, said inlet port and said outlet port provided on said at least a portion of said extension.

34. The artificial disc of claim 33, wherein said extension is formed integral with the artificial disc.

35. The artificial disc of claim 33, wherein said extension is selectively removable from the remainder of the artificial disc.

36. The artificial disc of claim 30, wherein said peripheral region has an outer profile substantially corresponding to the size and shape of the intervertebral disc space.

37. The artificial disc of claim 30, wherein said expanded configuration of said expandable region defines a pair of spherical-shaped protrusions extending in opposite directions and positionable within spherical-shaped recesses formed in respective ones of the adjacent vertebrae.

38. The artificial disc of claim 30, wherein said an expandable region comprises a non-porous flexible bladder defining said interior chamber.

39. The artificial disc of claim 30, wherein said expansion media is selected from the group consisting of a resin/catalyst mixture, a polymer-based material, a bone cement, a saline solution and water.

40. A method for implanting an artificial disc within an intervertebral disc space between adjacent vertebrae, comprising:
providing an artificial disc including an inner expandable region and an outer peripheral region, the expandable region comprising a non-porous flexible bladder that is transitionable from an initial configuration to an expanded configuration, the peripheral region having upper and lower surfaces defining a height substantially equal to the height of the intervertebral disc space;
forming a recess in a vertebral endplate of at least one of the adjacent vertebrae;
inserting the artificial disc into the intervertebral disc space with the upper and lower surfaces of the peripheral region facing respective ones of the adjacent vertebrae and with the inner region generally aligned with the recess in the vertebral endplate; and
introducing an expansion media into the non-porous flexible bladder to transition the inner region to the expanded configuration with the expanded inner region positioned within the recess in the vertebral endplate.

41. The method of claim 40, wherein the expansion media is selected from the group consisting of a resin/catalyst mixture, a polymer-based material, a bone cement, a saline solution and water.

42. The method of claim 40, further comprising removing at least a portion of the expansion media from the non-porous flexible bladder.

43. The method of claim 42, further comprising providing a vacuum source in communication with the non-porous flexible bladder to facilitate the removing of the at least a portion of the expansion media from the non-porous flexible bladder.

44. The method of claim 42, wherein the artificial disc includes an inlet port and an outlet port, the inlet port in communication with the non-porous flexible bladder for the introducing of the expansion media into the non-porous flexible bladder, the outlet port in communication with the non-porous flexible bladder for the removing of the at least a portion of the expansion media from the non-porous flexible bladder.

45. The method of claim 40, wherein the inserting of the artificial disc into the intervertebral disc space occurs while the inner region is in the initial configuration.

46. The method of claim 40, wherein the outer peripheral region includes a posterior surface defining a concave curvature; and
wherein the method further comprises positioning the concave curvature adjacent a posterior region of the intervertebral disc space.

47. The method of claim 40, wherein the expandable region defines at least one spherical-shaped protrusion when transitioned to the expanded configuration; and
the method further comprising positioning the at least one spherical-shaped protrusion within the recess formed in the vertebral endplate of the at least one of the adjacent vertebrae.

48. The method of claim 47, wherein a recess is formed in a vertebral endplate of each adjacent vertebrae, the expandable region defining a pair of spherical-shaped protrusions extending in opposite directions when transitioned to the expanded configuration; and
the method further comprising positioning the pair of spherical-shaped protrusions within respective ones of the recesses formed in the vertebral endplates of the adjacent vertebrae.

49. The method of claim 40, wherein the height of the peripheral region remains substantially unchanged during the introducing of the expansion media into the non-porous flexible bladder.

50. An instrument for preparing first and second vertebrae for receipt of an artificial disc within the intervertebral disc space between the vertebrae, comprising:
a distal end portion positionable within the intervertebral disc space and a handle portion extending from said distal end portion and positionable outside of the intervertebral disc space, said distal end portion including a ball-shaped cutting portion and an annular ring portion extending about a periphery of said ball-shaped cutting portion, said ball-shaped cutting portion including a first cutting element configured to form a first spherical-shaped recess in the first vertebra and a second cutting element configured to form a second spherical-shaped recess in the second vertebra.
